(19) United States
(12) Patent Application Publication
(10) Pub. No.: US 2005/0106220 A1
(43) Pub. Date: May 19, 2005

(54) Agent for improving mobility and general health of senior companion animals

(76) Inventors: Kentaro Inagawa, Kawasaki (JP); Makoto Bannai, Kawasaki (JP); Shinobu Seki, Kawasaki (JP); Norio Kogure, Kawasaki (JP)

Correspondence Address: AJINOMOTO CORPORATE SERVICES, LLC;INTELLECTUAL PROPERTY DEPARTMENT, 1120 CONNECTICUT AVE., N.W., WASHINGTON, DC, 20036, US

(21) Appl. No.: 10/969,870

(22) Filed: Oct. 22, 2004

(30) Foreign Application Priority Data
Oct. 29, 2003 (JP) NO.2003-368320

(51) Int. Cl. A61K 31/5513; A61K 31/495; A61K 31/195

(52) U.S. Cl. 424/442; 514/561; 514/221

(57) ABSTRACT

An agent for improving mobility and general health of senior companion animals is provided whereby said agent contains one or more substances selected from the group consisting of a GABA_{A} receptor agonist, a GABA_{B} receptor agonist and a GABA_{A} receptor enhancing substance as an active ingredient. Compositions containing said agent, including a pet food additive and a pet food, as well as methods of administering said compositions to senior companion animals are also provided.

### BACKGROUND OF THE INVENTION

1. Field of the Invention

The present invention relates to an agent for improving the general health and mobility of senior companion animals, a composition containing the agent, a pet food additive containing said agent, and a pet food containing said agent.

2. Brief Description of the Related Art

A recent increase in the popularity of pets and the need for companion animals has resulted in increased breeding of these animals. Furthermore, the advancement of veterinary science has lead to increased life spans for these animals. Accordingly, experts in the veterinary field have increasingly observed and reported problems which typically occur as these animals grow older (see Journal of the American Veterinary Medical Association, Vol. 218, No. 11, Pages 1787-1791 (2001), and Journal of the American Veterinary Medical Association, Vol. 218, No. 11, Pages 1792-1795 (2001)). These problems include difficulty breathing, forced or labored breathing, leg trembling, difficulty walking, decreased reaction times, decreased excretion, failure of one or more of the five senses, loss of directional sense, inactivity, and the like. Furthermore, the owners of older pets are faced with increasing mental and physical burdens on a daily basis. Pet foods formulated specifically for older dogs and cats are available, and typically contain antioxidants, vitamins and the like, as well as other additives which typically address decreased brain function (see Hill's Prescription Diet (for canine) b/d pamphlet).

Bannai et al. has reported that a GABA receptor agonist exhibits a running neuron inhibitory activity, which results in a poriomania inhibition effect, as well as an idle barking inhibition effect (see U.S. Pat. No. 6,380,176, Journal of the American Veterinary Medical Association, Vol. 218, No. 11, Pages 1787-1791 (2001), and Journal of the American Veterinary Medical Association, Vol. 218, No. 11, Pages 1792-1795 (2001). However, the effect of a GABA receptor agonist on senior companion animals is unknown. See also U.S. Pat. No. 6,380,176 and U.S. Patent Application Publication No. 2003-0018027.

Pet foods designed for older animals are typically expensive due to high unit prices of various materials. In addition, these pet foods are typically formulated to prevent the decline of the overall brain function. Accordingly, an agent, composition, pet food additive, or pet food designed for the older animal which is relatively inexpensive, fast-acting, and/or exhibits a different mechanism of action is desirable in the art.

### SUMMARY OF THE INVENTION

It is an object of the present invention to provide an agent for improving the general health and mobility of senior companion animals comprising one or more substances selected from the group consisting of a GABA_{B} receptor agonist, a GABA_{A} receptor agonist, and a GABA_{A} receptor enhancing substance.

It is a further object of the present invention to provide the agent as described above, wherein said GABA_{B} receptor agonist is GABA, baclofen or SKF 97541.

It is a further object of the present invention to provide the agent as described above, wherein the GABA_{A} receptor agonist is GABA, isoguvacine, muscimol or THIP.

It is a further object of the present invention to provide the agent as described above, wherein the GABA_{A} receptor enhancing substance is benzodiazepine.

It is a further object of the present invention to provide the agent as described above, wherein said agent is administered at a dose from 1 to 3,000 mg/kg body weight per day.

It is a further object of the present invention to provide the agent as described above, wherein said senior companion animal suffers from a condition selected from the group consisting of difficulty breathing, forced or labored breathing, leg trembling, difficulty walking, decreased reaction times, decreased excretion, failure of one or more of the five senses, loss of directional sense, and inactivity.

It is a further object of the present invention to provide a composition containing the the agent as described above.

It is a further object of the present invention to provide the composition as described above, wherein the composition also contains an amino acid and/or a vitamin.

It is a further object of the present invention to provide a pet food additive containing the compostion or agent as described above.

It is a further object of the present invention to provide a pet food containing the agent or composition as described above.

It is a further object of the present invention to provide a method for improving the general health and/or mobility of a senior companion animal comprising administering to said animal the agent, composition, and/or pet additive or food as described above, wherein said animal suffers from difficulty breathing, forced or labored breathing, leg trembling, difficulty walking, decreased reaction times, decreased excretion, failure of one or more of the five senses, loss of directional sense, and inactivity.

### BRIEF DESCRIPTION OF THE DRAWING

FIG. 1 shows the results of measuring an RR interval relative to the time following GABA administration.

### DETAILED DESCRIPTION OF THE PREFERRED EMBODIMENTS

In view of the foregoing objects and the unsolved problems in the art, the present inventors have conducted investigations, and have found that a GABA_{B} receptor agonist, a GABA_{A} receptor agonist, and a GABA_{A} receptor enhancing substance (these three substances are termed “GABA receptor agonist”) can improve the mobility and general overall health of senior companion animals.

The present inventors have studied the influence of the GABA receptor agonist on the sympathetic and parasympathetic nerve, and have found that the GABA receptor agonist can activate the sympathetic nerve. In view of this result, the possibility that the GABA receptor agonist can have a positive effect on the general health and mobility of senior companion animals was investigated by administering the GABA receptor agonist to senior dogs. As a result, the present inventors found that various conditions which are typically present in senior dogs were improved upon administration of the GABA receptor agonist. Although it has been reported that the GABA receptor agonist has an activity of inhibiting unintentional motion of animals, or poriomania, (see U.S. Pat. No. 6,380,176 and US 2003-0018027), it has now been surprisingly found that the GABA receptor agonist can increase mobility and activity of these animals.

The agent of the present invention can be administered either alone, as a composition containing the agent, as a pet food additive, or as a pet food containing the agent. When administered in any of the above forms, the agent is able to improve one or more of the conditions which typically occur in older animals, and include the following: difficulty breathing, forced or labored breathing, leg trembling, difficulty walking, decreased reaction times, decreased excretion, failure of one or more of the five senses, loss of directional sense, inactivity, and the like.

The senior companion animals of the present invention include mammals, preferably dogs, cats, rodents and the like. Animals which are breed as pets, as well as those that are trained for assistance with the disabled are encompassed. Domestically-bred dogs are the most preferred.

The age of the senior companion animal as defined by the present invention is preferably 7 years and older

Conditions which typically occur in senior companion animals as defined by the present invention include difficulty breathing, forced or labored breathing, leg trembling, difficulty walking, decreased reaction times, decreased excretion, failure of one or more of the five senses, loss of directional sense, inactivity, and the like.

The GABA receptor agonist useful as an agent of the present invention is described below. The GABA receptor agonist is not particularly limited so long as it is capable of acting on the GABA receptor. GABA (γ-aminobutyric acid) is preferable. Specific examples of the GABA_{B} receptor agonist include, but are not limited to GABA, baclofen (4-amino-3-(4-chlorophenyl)butanoic acid) and SKF 97541 (3-aminopropyl(methyl)phosphinic acid), and the like. Specific examples of the GABA_{A} receptor agonist include, but are not limited to GABA, isoguvacine (1,2,3,6-tetrahydro-4-pyridinecarboxylic acid), muscimol (5-aminomethyl-3-hydroxyisoxazole), THIP (4,5,6,7-tetrahydroisoxazolo[5,4-c]pyridin-3-ol), and the like. Specific examples of the GABA_{A} receptor enhancing substance include, but are not limited to, benzodiazepine and the like.

The dose of the agent of the present invention is from 1 to 3,000 mg/kg body weight per day, preferably from 1 to 300 mg/kg body weight per day, most preferably from 10 to 50 mg/kg body weight per day. When administering to a senior companion animal other than an oral route (for example, intraperitoneal injection), a smaller dose can be used.

The GABA receptor agonist may be formulated as a tablet, a capsule, a syrup, a powder, a suspension, or the like using appropriate additives, such as an excipient. The GABA receptor agonist may also be formulated without using any additives. Accordingly, the agent of the present invention includes not only a formulation containing the GABA receptor agonist and additives, but also the GABA receptor agonist alone.

The agent of the present invention can be formulated into a pet food additive by incorporating an amino acid, a vitamin and the like. The pet food additive of the present invention does not have to be added to a pet food, but can be directly administered to senior companion animals.

The amino acid which may be contained in the composition or pet food additive of the present invention, and its daily dosage are as follows: isoleucine—0 to 480 mg/kg body weight, leucine—0 to 840 mg/kg body weight, valine—0 to 600 mg/kg body weight, lysine—0 to 500 mg/kg body weight, methionine—0 to 300 mg/kg body weight, phenylalanine—0 to 860 mg/kg body weight, threonine—up to 440 mg/kg body weight, tryptophan—0 to 130 mg/kg body weight, histidine—0 to 500 mg/kg body weight, arginine—0 to 1,300 mg/kg body weight, tyrosine—0 to 150 mg/kg body weight, alanine—0 to 1,200 mg/kg body weight, aspartic acid—0 to 2,000 mg/kg body weight, glutamine—0 to 2,600 mg/kg body weight, glutamic acid—0 to 2,600 mg/kg body weight, serine—0 to 2,000 mg/kg body weight, glycine—0 to 700 mg/kg body weight, proline—0 to 900 mg/kg body weight, cystine—0 to 1,400 mg/kg body weight, cysteine—0 to 1,400 mg/kg body weight and sodium glutamate (MSG)—0 to 900 mg/kg body weight. However, when an amino acid is added to a pet food or a pet food additive, it is advisable to consider the existing contents of various amino acids in the pet food or additive and adjust the additional amount accordingly.

Vitamins which may be in the composition or pet food additive of the invention may include vitamin A, vitamin D, vitamin E, vitamin K, vitamin B₁, vitamin B₂, pantothenic acid, pyridoxine, cobalamin, folic acid, biotin, niacin, choline and the like. The recommended dietary allowances per day for dogs may be vitamin A: 110 to 1,100 IU/kg body weight, vitamin D: 11 to 110 IU/kg body weight, vitamin E: 1.1 to 11 IU/kg body weight, vitamin K: 24 to 240 IU/kg body weight, vitamin B₁: 22 to 220 μg/kg body weight, vitamin B₂: 48 to 480 μg/kg body weight, pantothenic acid: 220 to 2,200 μg/kg body weight, pyridoxine: 22 to 220 μg/kg body weight, cobalamin: 0.5 to 5 μg/kg body weight, folic acid: 4.0 to 40 μg/kg body weight, biotin: 2.2 to 22 μg/kg body weight, niacin: 250 to 2,500 μg/kg body weight and choline: 26 to 260 μg/kg body weight. However, when a vitamin is added to a pet food, or pet food additive similar to the amino acid additives above, it is advisable to consider the existing contents of various vitamins in the pet food or additive and adjust the additional amount accordingly.

The agent, or composition, or pet food additive of the present invention may be administered by adding it to the existing pet food, or by being formulated into the pet food.

When added to existing pet food, the added amount of the agent, or GABA receptor agonist, may be from 0.0001 to 1% by weight, preferably from 0.001 to 0.8% by weight, more preferably from approximately 0.01 to approximately 0.07% by weight, especially preferably from approximately 0.1 to approximately 0.5% by weight. In other words, when the agent of the present invention is added to existing pet food in such an amount to cause companion animals to take the same, it is advisable that the GABA receptor agonist is taken in an amount of from 0.1 to 3,000 mg/kg body weight, preferably from 1 to 300 mg/kg body weight per day, more preferably from 10 to 50 mg/kg body weight per day.

When being formulated as a composition of a pet food containing the agent of the presnt invention or the food additive, it is advisable to add these in appropriate amounts for a commercially available pet food. A pet food composition designed for senior dogs or senior cats is preferable. Specifically, a composition to be incorporated into a pet food for senior dogs may be protein: 10 to 25%, fat: 10 to 25%, carbohydrate: 50 to 60%, fibrous material: 1 to 10%, calcium: 0.1 to 1%, phosphorus: 0.1 to 1%, sodium: 0.01 to 0.2%, potassium: 0.1 to 1.5%, magnesium: 0.01 to 0.2%, and chloride: 0.1 to 1%. It is also possible to provide a composition which is obtained by incorporating a GABA receptor agonist into a pet food composition which also contains minor components such as taurine, carnitine, lipoic acid and β-carotene. The pet food containing an agent or composition of the present invention can be produced by ordinary methods.

When the agent for improving general health or mobility or the pet food additive or the pet food containing the agent or additive according to the present invention is administered to senior companion animals, it is advisable that once the administration is started and the effect of is identified, the administration be continued.

The agent, composition, pet food additive, or pet food of the present invention is administered to senior companion animals, and results in a remarkable improvement in general health conditions, including difficulty breathing, forced or labored breathing, leg trembling, difficulty walking, decreased reaction times, decreased excretion, failure of one or more of the five senses, loss of directional sense, inactivity. All of these conditions are common in dogs of advanced age.

As described above, the problems of senior companion animals can be solved by administering the agent of the present invention as such or in the form of a composition, pet food additive, or pet food.

### EXAMPLES

Hereinafter, the present invention will be explained more specifically with reference to the following non-limiting examples.

### Test Example 1

Three 10 month old Beagle dogs were confined in darkness from 19:00 (7pm) until 7:00 (7am). An electrocardiogramr was obtained under free movement using a Holter ECG recorder (wireless installation-type ECG recorder). An electrocardiogram was obtained every 30 minutes for 1 minute. A capsule containing 300 mg/kg body weight of GABA was orally administered to each dog just before the dark period. As a control, an empty capsule was administered.

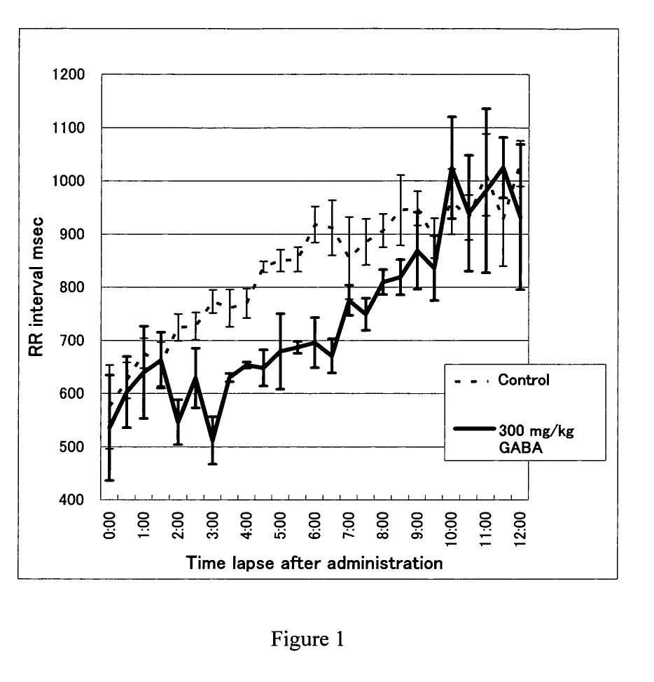

An RR interval (msec, inverse number of a heart rate) relative to the time that lapsed after administration was measured. As a result, the average RR interval was shortened by the administration of GABA for approximately 2 to 9 hours after the administration, and stimulus of the sympathetic nerve was observed (FIG. 1 to be illustrated later).

In the following Examples, one-tenth of the amount used in the above Test Example was used for safety's sake.

### Example 1

Dog food was mixed with GABA (30 mg/kg body weight). Mongrel dog A (17 years old) was fed this mixture once a day in the evening for 2 weeks. Consequently, the forced and labored breathing evident prior to the administration disappeared after administration of the GABA food mixture.

### Example 2

Dog food was mixed with GABA (30 mg/kg body weight). Mongrel dog B (14 years old) was fed this mixture once a day for 2 weeks. Consequently, leg trembling, which had been evident before the administration, disappeared. Also, the dog drank water on its own after the administration. Thus, improvement in mobility and general health was observed.

### Example 3

Dog food was mixed with GABA (30 mg/kg body weight). Toy poodle A (15 years old) was fed this mixture once a day for 2 weeks. Consequently, difficult breathing, which had been observed before the administration, was decreased. Also, the dog sought out his owner and wanted to stay by him. Thus, an improvement in the dog's sense of hearing and sense of smell, mobility and general health was observed.

### Example 4

Dog food was mixed with GABA (30 mg/kg body weight). Toy poodle B (12 years old) was fed this mixture once a day for 2 weeks. Although the dog had walked with tottering steps before the administration, it walked firmly with steady steps on the ground. Thus, an improvement in mobility was observed.

### Example 5

Dog food was mixed with GABA (30 mg/kg body weight). Pomeranian A (14 years old) was fed this mixture once a day for 2 weeks. Consequently, continuous sleeping all day long, which had been the dog's routine before the administration, was decreased, and the dog clearly showed signs that he wanted to go for a walk. Thus, an improvement in general health and activity level was observed.

### Example 6

Dog food was mixed with GABA (30 mg/kg body weight). Shiba Ken (Japanese breed) A (16 years old) was fed with this mixture once a day for 2 weeks. Consequently, the dog was able to move around easier and clearly with less pain than before the administration. Thus, an improvement in mobility was observed.

### Example 7

SKF 97541 was intravenously administered once to Shiba Ken B (15 years old) at a dose of from 0.01 to 0.1 mg/kg body weight. Consequently, the turning motion the dog exhibited before the administration was eliminated at any dose.

While the invention has been described in detail with reference to preferred embodiments thereof, it will be apparent to one skilled in the art that various changes can be made, and equivalents employed without departing from the scope of the invention. Each of the aforementioned documents, including the foreign priority document, JP2003-368320, is incorporated by reference herein in its entirety.

1. An agent for improving the general health and mobility of senior companion animals comprising one or more substances selected from the group consisting of a GABA_{B} receptor agonist, a GABA_{A} receptor agonist, and a GABA_{A} receptor enhancing substance.

2. The agent according to claim 1, wherein said GABA_{B} receptor agonist is selected from the group consisting of GABA, baclofen and SKF 97541.

3. The agent according to claim 1, wherein the GABA_{A} receptor agonist is selected from the group consisting of GABA, isoguvacine, muscimol and THIP.

4. The agent according to claim 1, wherein the GABA_{A} receptor enhancing substance comprises benzodiazepine.

5. The agent according to claim 1, wherein said agent is administered at a dose from 1 to 3,000 mg/kg body weight per day.

6. The agent according to claim 1, wherein said senior companion animal suffers from a condition selected from the group consisting of difficulty breathing, forced or labored breathing, leg trembling, difficulty walking, decreased reaction times, decreased excretion, failure of one or more of the five senses, loss of directional sense, and inactivity.

7. A composition comprising the agent of claim 1.

8. The composition of claim 7, further comprising an amino acid.

9. The composition of claim 8, further comprising a vitamin.

10. The composition of claim 7, which is a pet food additive.

11. The composition of claim 7, which is a pet food.

12. A pet food additive comprising the agent of claim 1.

13. A pet food comprising the agent of claim 1.

14. A method for improving the general health of a senior companion animal comprising administering to said animal the agent of claim 1.

15. A method for improving the mobility of a senior companion animal comprising administering to said animal the agent of claim 1.

16. A method for improving the general health of a senior companion animal comprising administering the pet food of claim 13.

17. The method of claim 14, wherein said agent is administered at a dose from 1 to 3,000 mg/kg body weight per day.

18. The method of claim 14, wherein said animal suffers from a condition selected from the group consisting of difficulty breathing, forced or labored breathing, leg trembling, difficulty walking, decreased reaction times, decreased excretion, failure of one or more of the five senses, loss of directional sense, and inactivity.
